(19) United States
(12) Patent Application Publication
(10) Pub. No.: US 2026/0041618 A1
(43) Pub. Date: Feb. 12, 2026

(54) A LIQUID CLEANSING COMPOSITION

(71) Applicant: Conopco, Inc., d/b/a UNILEVER, Englewood Cliffs, NJ (US)

(72) Inventors: Shajahan ABDUL KAREEM, Pondicherry (IN); Megan Ruth JANKOVSKY, Stratford, CT (US); Teanoosh MOADDEL, Watertown, CT (US); Shailendra PRATAP, Bangalore (IN); Saswati PUJARI, Bangalore (IN)

(73) Assignee: Conopco, Inc., d/b/a UNILEVER, Englewood Cliffs, NJ (US)

(21) Appl. No.: 19/099,192

(22) PCT Filed: Jul. 21, 2023
(86) PCT No.: PCT/EP2023/070333

§ 371 Date: Jan. 28, 2025

(30) Foreign Application Priority Data
Aug. 4, 2022 (EP) 22188808.4

(51) Int. Cl. A61K 8/36; A61K 8/362; A61K 8/365; A61K 8/368; A61K 8/46; A61Q 19/10

(52) U.S. Cl. CPC A61K 8/361; A61K 8/362; A61K 8/365; A61K 8/368; A61K 8/463; A61K 8/466; A61Q 19/10; A61K 2800/30; A61K 2800/596; A61K 2800/805

(57) ABSTRACT

The present invention relates to a liquid cleansing composition. Particularly, the present invention relates to a liquid cleansing composition that provides improved deposition of an active, an octadecanoic acid, when applied on to a surface. The composition comprises an octadecanoic acid and an organic acid with pKa from 2.5 to 4.9 wherein the organic acid is other than the octadecanoic acid.

### FIELD OF THE INVENTION

The present invention relates to a cleansing composition. Particularly, the present invention relates to a liquid cleansing composition that provides improved deposition of benefit agents.

### BACKGROUND OF THE INVENTION

Several liquid cleansing compositions are known in the art that contain one or the other benefit also called as actives. Such benefit agents or actives are often included in a liquid cleansing composition with an intention to deliver a benefit. For example, a moisturizing agent is included in cleansing compositions e.g. a handwash composition and a bodywash composition, to deliver a moisturizing effect.

However, the technical effect to be achieved through including an active in a cleansing composition tends to be limited due to a variety of reasons. One of the possible reasons is said to be deposition of an active on to a surface to be cleansed or washed. In general, deposition of an active on to a surface through a cleansing composition is challenging as the cleansing action generally includes a step of removing the composition (washing it off) from the surface, typically within a few seconds to a few minutes after application. Therefore, depositing an active on to a surface in limited duration is very challenging.

Therefore, need still exists to provide a liquid cleansing composition that provides improved deposition of benefit agents e.g. octadecanoic acids. Examples of octadecanoic acids include 10-hydroxystearic acid, 12-hydroxystearic acid, petroselinic acid, and conjugated linoleic acids.

The present invention has found that a liquid cleansing composition that comprises select amounts of octadecanoic acid in combination with select amounts of organic acids with certain pKa range, wherein these organic acids with certain pKa range are organic acids other than the octadecanoic acid, provided improved deposition of the octadecanoic acid. For example, when an octadecanoic acid e.g. 12-hydroxystearic acid (12-HSA), is used in combination with an organic acid with pKa in the range from 2.5 to 4.9, such as benzoic acid (pKa=4.2), in a liquid cleansing composition, it resulted in improved deposition of 12-HSA on to a surface e.g. skin.

### SUMMARY OF THE INVENTION

Accordingly, in a first aspect, the present invention relates to a liquid cleansing composition comprising:
a. from 0.01 to 2 wt %, preferably 0.1 to 2 wt %, octadecanoic acid; and
b. from 0.01 to 5 wt %, preferably 0.5 to 5 wt %, organic acid with pKa in the range from 2.5 to 4.9,
   wherein the organic acid is other than the octadecanoic acid;
   wherein the octadecanoic acid is selected from 10-hydroxystearic acid, 12-hydroxystearic acid, petroselinic acid, conjugated linoleic acid and mixtures thereof and preferably is not stearic acid.

In a second aspect, the present invention relates to a method of improving deposition of an octadecanoic acid comprising the steps:
i. applying the composition of the first aspect on to a surface; and
ii. at least partially removing the composition from the surface,
   wherein the step of removing the composition is carried out within 5 minutes of the step of applying the composition on to the surface;
   wherein the octadecanoic acid is selected from 10-hydroxystearic acid, 12-hydroxystearic acid, petroselinic acid, conjugated linoleic acid and mixtures thereof and preferably is not stearic acid.

In a third aspect, the present invention relates to use of organic acids with pKa in the range from 2.5 to 4.9, in a liquid cleansing composition comprising an octadecanoic acid for improving deposition of the octadecanoic acid on to a surface wherein the organic acids with pKa in the range from 2.5 to 4.9, are organic acids other than the octadecanoic acid;

wherein the octadecanoic acid is selected from 10-hydroxystearic acid, 12-hydroxystearic acid, petroselinic acid, conjugated linoleic acid and mixtures thereof and preferably is not stearic acid.

### DETAILED DESCRIPTION OF THE INVENTION

Any feature of one aspect of the present invention may be utilized in any other aspect of the invention. The word “comprising” is intended to mean “including” but not necessarily “consisting of” or “composed of.” In other words, the listed steps or options need not be exhaustive. Except in the operating and comparative examples, or where otherwise explicitly indicated, all numbers in this description indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated. Unless specified otherwise, amounts as used herein are expressed in percentage by weight based on total weight of the composition and may be abbreviated as “wt %”. The use of any and all examples or exemplary language e.g. “such as” provided herein is intended merely to better illuminate the invention and does not in any way limit the scope of the invention otherwise claimed.

Accordingly, in a first aspect, the present invention relates to a liquid cleansing composition comprising:
a. from 0.01 to 2 wt %, preferably 0.1 to 2 wt %, octadecanoic acid; and
b. from 0.01 to 5 wt %, preferably 0.5 to 5 wt %, organic acid with pKa in the range from 2.5 to 4.9,
wherein the organic acid is other than the octadecanoic acid;
wherein the octadecanoic acid is selected from 10-hydroxystearic acid, 12-hydroxystearic acid, petroselinic acid, conjugated linoleic acid and mixtures thereof and preferably is not stearic acid.

### A Liquid Cleansing Composition

The present invention relates to a liquid cleansing composition (the composition) comprising from 0.01 to 2 wt %, preferably 0.1 to 2 wt %, octadecanoic acid; and from 0.01 to 5 wt %, preferably 0.5 to 5 wt %, organic acid with pKa in the range from 2.5 to 4.9, wherein the organic acid is other than the octadecanoic acid. The octadecanoic acid is selected from 10-hydroxystearic acid, 12-hydroxystearic acid, petroselinic acid, conjugated linoleic acid and mixtures thereof and preferably is not stearic acid.

The composition is preferably applied on to a surface that may be an inanimate or animate surface. More preferably, the composition is applied to an animate substrate e.g. human skin. ‘Skin’ as used herein, preferably means to include skin on any part of the body e.g. face, neck, chest, back, arms, underarms, hands, legs, buttocks and scalp. The composition is typically used with an appropriate amount of water which typically creates lather.

Preferably, the viscosity of the composition is in the range from 2500 to 4500 cps, more preferably from 3000 to 3500 cps at 25° C. Products with such viscosity are generally referred to as liquid compositions as they are free flowing and pourable. Such liquid compositions are distinct from gels and creams which have a semi-solid consistency with much higher viscosity.

### Octadecanoic Acid

The composition comprises octadecanoic acid. It will be understood that the octadecanoic acid is an acid that contains 18 carbon atoms in a chain that may be linear or branched; and may be saturated or unsaturated at the same time. The octadecanoic acid as per the present invention may have a substitution e.g. a hydroxy (—OH) group, on the carbon chain.

The octadecanoic acid is selected from 10-hydroxystearic acid (10-HSA), 12-HSA, petroselinic acid, conjugated linoleic acid and mixtures thereof. More preferably, the octadecanoic acid is selected from 10-HSA, 12-HSA, petroselinic acid and mixtures thereof. Even more preferably, the octadecanoic acid is selected from 10-HSA, 12-HSA and mixtures thereof. Further more preferably, the octadecanoic acid selected is 12-HSA. The octadecanoic acid is preferably not stearic acid.

The composition comprises from 0.01 to 2 wt %, preferably from 0.05 to 1.5 wt %, more preferably from 0.1 to 2 wt %, even more preferably from 0.1 to 1 wt %, even more preferably from 0.15 to 1 wt %, even more preferably from 0.25 to 0.75 wt % and further more preferably from 0.3 to 0.5 wt % octadecanoic acid.

Organic Acid with pKa in the Range from 2.5 to 4.9 Wherein the Organic Acid is Other than the Octadecanoic Acid

The composition comprises an organic acid with pKa in the range from 2.5 to 4.9, more preferably 2.5 to 4.7, and even more preferably from 2.9 to 4.5, which is other than the octadecanoic acid.

It will be understood that the organic acid with pKa in the range from 2.5 to 4.9 is an organic acid other than the octadecanoic acid described above. Preferably, the organic acid with pKa in the range from 2.5 to 4.9 does not comprise any octadecanoic acid. Preferably the octadecanoic acid is not stearic acid.

Preferably, these organic acids with pKa in the range from 2.5 to 4.9 are selected from lactic acid (pKa=3.86), citric acid (pKa=3.13), glycolic acid (pKa=3.83), benzoic acid (pKa=4.2), octanoic acid (also known as caprylic acid; pKa=4.89), succinic acid (pKa=4.2), salicylic acid (also known as 2-hydroxybenzoic acid; pKa=2.97), butyric acid (pKa=4.82), phenylpropionic acid (pKa=4.66) and mixtures thereof; more preferably from lactic acid, glycolic acid, benzoic acid and mixtures thereof; and even more preferably the organic acid other than the octadecanoic acid having pKa in the range from 2.5 to 4.9 is benzoic acid.

The composition comprises from 0.01 to 5 wt %, preferably from 0.05 to 4 wt %, more preferably from 0.1 to 3 wt %, even more preferably from 0.5 to 3 wt %, and even more preferably from 1 to 2 wt % of these other organic acids.

### Anionic Surfactant

Preferably, the composition further comprises an anionic surfactant selected from alkyl sulfates, alkyl ether sulfates (AES), alpha olefin sulfonates (AOS) and mixtures thereof. More preferably, an anionic surfactant is selected from alkyl sulfates, alkyl ether sulfates and mixtures thereof. Even more preferably, alkyl ether sulfates are used as the anionic surfactant in the composition. Anionic surfactants are known to provide foam and cleansing action.

Examples of alkyl sulfates that may be used as anionic surfactant in the composition include sodium lauryl sulfate (SLS), sodium myristyl sulfate and mixtures thereof.

Examples of AES that may be used as an anionic surfactant include anionic surfactants of the general formula:

R₁—(OR′)ₙ—O—SO₃⁻M⁺, wherein:
R₁ is saturated or unsaturated C₈-C₁₆, preferably C₁₂-C₁₄ alkyl chain; preferably, R₁ is a saturated C₈-C₁₆, more preferably a saturated C₁₂-C₁₄ alkyl chain;
R′ is ethylene; n is from 1 to 18; preferably from 1 to 15, more preferably from 1 to 10 and even more preferably from 1 to 5,
M⁺ is a suitable cation which provides charge neutrality, preferably sodium, calcium, potassium, or magnesium, more preferably a sodium cation.

Examples of AES that may be used as anionic surfactant in the composition include sodium lauryl ether sulfate (SLES), sodium myristyl ether sulfate and sodium palmityl ether sulfate and mixtures thereof. Preferred AES is SLES having 1 to 3 ethylene oxide units per molecule. SLES having 1 to 2 ethylene oxide units per molecule is more preferred.

Preferably, the composition comprises from 1 to 20 wt %, more preferably from 3 to 18 wt %, even more preferably from 5 to 15 wt %, further more preferably from 8 to 14 wt %, still more preferably from 10 to 12 wt % anionic surfactant that may be selected from one or more anionic surfactants described above.

### Amphoteric Surfactant

Preferably, the composition further comprises an amphoteric surfactant. They provide foam boost and improve sensorial of the composition. Preferably, amphoteric surfactants are selected from cocamidopropyl betaine (CAPB), cocamide monoethanolamide (CMEA), cocoamphoacetate and mixtures thereof.

Preferably, the composition comprises from 1 to 5 wt %, more preferably from 1.5 to 4.5 wt %, even more preferably from 2 to 4 wt %, further more preferably from 2 to 3.5 wt % and still more preferably from 2.5 to 3 wt % amphoteric surfactants.

### pH of the Composition

Preferably, the pH of the composition is in the range from 3 to 10, more preferably, from 3.5 to 9, even more preferably from 4 to 9, further more preferably from 4 to 8, still more preferably from 4 to 7, yet more preferably from 4 to 7.5, still further more preferably from 4 to 7, yet further more preferably from 4 to 6.5, even further more preferably from 4 to 6 and yet more further preferably from 4 to 5. The pH may be adjusted using know pH adjusting agents e.g. sodium hydroxide and hydrochloric acid.

Preferably, the composition comprises 0 to 0.001 wt % soap (salt of a fatty acid). More preferably, the composition comprises 0 wt % soap, i.e. it does not comprise soap in any amount, or is soap-free.

### Polymers

Preferably, the composition further comprises water soluble/dispersible polymers. These polymers can be cationic, anionic, amphoteric or nonionic types with molecular weights higher than 100,000 Dalton. They are known to increase the viscosity and stability of liquid cleansing compositions, to enhance in-use and after-use skin sensory feels, and to enhance lather creaminess and lather stability. Such polymers may preferably be used in amounts from 0.1 to 10 wt %, more preferably from 0.1 to 5 wt %, even more preferably from 0.25 to 3 wt % and further more preferably from 0.5 to 2 wt %

Examples of water soluble/or dispersible polymers include the carbohydrate gums such as cellulose gum, microcrystalline cellulose, cellulose gel, hydroxyethyl cellulose, hydroxypropyl cellulose, hydroxypropyl methyl cellulose, sodium carboxymethylcellulose, methyl cellulose, ethyl cellulose, guar gum, gum karaya, gum tragacanth, gum arabic, gum acacia, gum agar, xanthan gum and mixtures thereof; modified and nonmodified starch granules and pregelatinized cold water soluble starch; emulsion polymers such as Aculyn® 28, Aculyn® 22 or Carbopol® Aqua SF1; cationic polymer such as modified polysaccharides including cationic guar available from Rhone Poulenc under the trade name Jaguar® C13S, Jaguar® C14S, Jaguar® C17, or Jaguar® C16; cationic modified cellulose such as UCARE® Polymer JR 30 or JR 40 from Amerchol; N-Hance® 3000, N-Hance® 3196, N-Hance® GPX 215 or N-Hance® GPX 196 from Hercules; synthetic cationic polymer such as Merquat® 100, Merquat® 280, Merquat® 281 and Merquat® 550 sold by Nalco; cationic starches such as StaLok® 100, 200, 300 and 400 sold by Staley Inc.; cationic galactomannans such as Galactasol® 800 series by Henkel, Inc.; Quadrosoft® LM-200; and Polyquaternium-24®. Also suitable are high molecular weight polyethylene glycols such as Polyox® WSR-205 (PEG 14M), Polyox® WSR-N-60K (PEG 45), and Polyox® WSR-301 (PEG 90M).

Preferably, the composition further comprises polymers selected from vinyl Pyrrolidone/vinyl acetate copolymer.

### Water

Preferably, the composition comprises water from 60 to 98 wt %, more preferably from 65 to 95 wt %, even more preferably from 70 to 90 wt %, further more preferably from 75 to 85 wt % and still more preferably from 80 to 85 wt % water.

### Skin Lightening Agents

Preferably, the composition further comprises one or more skin lightening agents. Preferably, the skin lightening agent may be selected from ammonium lactate, arbutin, azelaic acid, kojic acid, butyl hydroxy anisole, butyl hydroxy toluene, citrate esters, 3 diphenyl propane derivatives, ellagic acid, gluco pyranosyl-1-ascorbate, gluconic acid, hydroquinone, 4 hydroxyanisole, linoleic acid, magnesium ascorbyl phosphate, 2,4 resorcinol derivatives, 3,5 resorcinol derivatives, vitamins like vitamin B3, vitamin B6, vitamin B12, vitamin C, vitamin A, resorcinol derivatives and mixtures thereof.

### Preservatives

Preferably, the composition further comprises preservatives to protect the composition against the growth of potentially harmful microorganisms. Particularly preferred preservatives include hydantoin derivatives, propionate salts, a variety of quaternary ammonium compounds, phenoxyethanol, imidazolidinyl urea, sodium dehydroacetate and benzyl alcohol. Preservatives are preferably used in amounts ranging from 0.01 wt % to 2 wt % and more preferably from 0.1 to 1 wt %.

### Emollients

The composition may further comprise emollients, such as stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate, myristyl myristate.

### Optional Ingredients

The compositions may further comprise a wide range of other optional components e.g. antioxidants, biological additives, buffering agents, colorants, astringents, fragrance, humectants, opacifying agents, conditioners, pH adjusters, skin soothing agents and skin healing agents.

In a second aspect, the present invention relates to a method of improving deposition of an octadecanoic acid comprising the steps:
i. applying the composition on to a surface; and
ii. removing the composition at least partially from the surface,
   wherein the step of removing the composition is carried out within 5 minutes of the step of applying the composition on to the surface. Preferably, the second step is carried out within 4 minutes, more preferably within 3 minutes, even more preferably within 2 minutes e.g. 1 minute, 30 seconds and 15 seconds.

When the method as descried in the second aspect is carried out, improved deposition of an octadecanoic acid is obtained on to a surface. Preferably, the method is non-therapeutic.

In a third aspect, the present invention relates to use of organic acids with pKa in the range from 2.5 to 4.9, in a liquid cleansing composition comprising an octadecanoic acid for improving deposition of the octadecanoic acid on to a surface wherein the organic acids with pKa in the range from 2.5 to 4.9 are organic acids other than the octadecanoic acid. Preferably, the use is non-therapeutic. For example, when an octadecanoic acid e.g. 12-hydroxystearic acid (12-HSA), is used in combination with an organic acid with pKa in the range from 2.5 to 4.9 such as benzoic acid (pKa=4.2), in a liquid cleansing composition, it resulted in improved deposition of 12-HSA on to a surface e.g. skin.

The invention is further described with reference to the following non-limiting examples:

### Examples

### Examples A, 1-9

### Protocols

Briefly, compositions as shown in table 1 below were prepared using differing concentrations of 12-HSA and Benzoic acid (as shown in table 1) in surfactant solution at 85° C. followed by cooling it down to 25° C.; and pH was adjusted to a desired value.

### Deposition Assay was Carried Out as Described Below:

A model skin sample, vitro skin, (Vitro Skin N-19 from IMS Testing Group) was cut into 5 cm×5 cm. It was was prewetted with 500 μL DI water and rubbed for 30 sec wearing gloves and excess water was drained by shaking. 80 mg of neat compositions (as shown in the table 1 below) was applied onto the vitro skin. It was lathered for 20 seconds with a fingertip wearing latex gloves. Lather retention was done for 30 seconds onto Vitro skin. Thereafter, vitro skin was rinsed off on both sides using 100 mL distilled water for about 30 seconds. The active deposited onto vitro skin was extracted in methanol by sonicating in bath sonicator for 20 minutes. The extract was analysed using LC-MS. Briefly, compositions shown in table 1 below were prepared using differing concentrations of 12-HSA and Benzoic acid (as shown below) in surfactant solution at 85° C. followed by cooling it down to 25° C.; and pH was adjusted to a desired value.

TABLE 1

| | 12-HSA | Benzoic acid | SLES 1EO | CAPB | NaCl | Deposition |
|---|---|---|---|---|---|---|
| Ex. | (wt %) | (wt %) | (wt %) | (wt %) | (wt %) | (μg/cm²) |
| A | 0.10 | — | 6.30 | 1.50 | 0.25 | 0.10 |
| 1 | 0.10 | 0.50 | 6.30 | 1.50 | 0.25 | 0.10 |
| 2 | 0.15 | 0.50 | 6.30 | 1.50 | 0.25 | 0.15 |
| 3 | 0.20 | 0.50 | 6.30 | 1.50 | 0.25 | 0.19 |
| 4 | 0.25 | 0.50 | 6.30 | 1.50 | 0.25 | 0.20 |
| 5 | 0.30 | 0.50 | 6.30 | 1.50 | 0.25 | 0.17 |
| 6 | 0.10 | 2.00 | 6.30 | 1.50 | 0.25 | 0.14 |
| 7 | 0.30 | 2.00 | 6.30 | 1.50 | 0.25 | 0.24 |
| 8 | 0.10* | 0.50 | 6.30 | 1.50 | 0.25 | 0.15 |
| 9 | 0.30* | 0.50 | 6.30 | 1.50 | 0.25 | 0.43 |

Note:
[pH = 4.0 except for * where pH = 9.0; water to 100]

### Examples B. 10-12

Further compositions were prepared as shown in Table-2 below and the deposition of 12HSA was assessed using the same procedure as used in the preceding examples of Table-1. The data is summarized in Table-2 below:

TABLE 2

| | | | SLES | | | |
|---|---|---|---|---|---|---|
| | 12-HSA | | 1EO | CAPB | NaCl | Deposition |
| Ex. | (wt %) | Organic acid | (wt %) | (wt %) | (wt %) | (μg/cm²) |
| A | 0.10 | — | 6.30 | 1.50 | 0.25 | 0.10 |
| B | 0.10 | 1.42% lauric acid + | 6.30 | 1.50 | 0.25 | 0.082 |
| | | 1.96% myristic acid + | | | | |
| | | 0.54% stearic acid | | | | |
| 10 | 0.10 | 2% phenyl propionic | 6.30 | 1.50 | 0.25 | 0.12 |
| | | acid | | | | |
| 11 | 0.10 | 1% benzoic acid + | 6.30 | 1.50 | 0.25 | 0.112 |
| | | 1% phenyl propionic | | | | |
| | | acid | | | | |
| 12 | 0.10 | 2% benzoic acid + | 6.30 | 1.50 | 0.25 | 0.167 |
| | | 1% phenyl propionic | | | | |
| | | acid | | | | |

The data in the table-2 above indicate that organic acids as per the invention (pKa 2.5 to 4.9) (Examples 10 to 12) singly or in combination, provide superior deposition of octadecanoic acid as defined by the invention, compared to organic acids with higher pKa (lauric and myristic acid), (Example-B).

1. A liquid cleansing composition comprising:
a. from 0.15 to 1 wt % of an octadecanoic acid; and
b. from 0.5 to 5 wt % organic acid with a pKa the range from 2.5 to 4.9;
wherein the organic acid is other than the octadecanoic acid;
wherein the octadecanoic acid is selected from 10-hydroxystearic acid, 12-hydroxystearic acid, petroselinic acid, conjugated linoleic acid and mixtures thereof and is not stearic acid;
wherein the composition comprises 1 to 20 wt % of an anionic surfactant; and
wherein the organic acid with pKa in the range from 2.5 to 4.9 is selected from lactic acid, citric acid, glycolic acid, benzoic acid, octanoic acid, succinic acid, salicylic acid, butyric acid, phenyl propionic acid; and mixtures thereof.

2. The liquid cleansing composition as claimed in claim 1, wherein the octadecanoic acid is selected from 10-hydroxystearic acid, 12-hydroxystearic acid, petroselinic acid, and mixtures thereof.

3. The liquid cleansing composition as claimed in claim 1, wherein the composition comprises octadecanoic acid in an amount from 0.15 to 0.5 wt %-% of the composition.

4. The liquid cleansing composition as claimed in claim 3, wherein the composition comprises organic acid with a pKa the range from 2.5 to 4.9 in an amount from 0.5 to 3 wt % of the composition.

5. The liquid cleansing composition as claimed in claim 1, wherein the octadecanoic acid is 12-hydroxystearic acid.

6. (canceled)

7. The liquid cleansing composition as claimed in claim 1, wherein the organic acid with the pKa in the range from 2.5 to 4.9 is selected from lactic acid, glycolic acid, benzoic acid and mixtures thereof.

8. The liquid cleansing composition as claimed in claim 1, wherein the organic acid with the pKa in the range from 2.5 to 4.9 is benzoic acid.

9. The liquid cleansing composition as claimed in claim 1, wherein the anionic surfactant is selected from alkyl sulfates, alkyl ether sulfates, alpha olefin sulfonates and mixtures thereof.

10. The liquid cleansing composition as claimed in claim 9, wherein the anionic surfactant is selected from alkyl sulfates, alkyl ether sulfates and mixtures thereof.

11. The liquid cleansing composition as claimed in claim 1, wherein the composition further comprises from 1 to 5 wt % amphoteric surfactant.

12. The liquid cleansing composition as claimed in claim 1, wherein a pH of the composition is in the range from 3 to 10.

13. The liquid cleansing composition as claimed in claim 1, wherein the composition does not contain soap.

14. A method of improving deposition of an octadecanoic acid comprising the steps:
i. applying the liquid cleansing composition as claimed in claim 1 on to a surface; and
ii. at least partially removing the liquid cleansing composition from the surface, wherein the step of removing the composition is carried out within 5 minutes of the step of applying the composition on to the surface;
wherein the octadecanoic acid is selected from 10-hydroxystearic acid, 12-hydroxystearic acid, petroselinic acid, conjugated linoleic acid and mixtures thereof and is not stearic acid.

15. (canceled)

16. The liquid cleansing composition as claimed in claim 12, wherein the pH is from 4 to 7.
